(19) United States
(12) Patent Application Publication
(10) Pub. No.: US 2020/0011845 A1
(43) Pub. Date: Jan. 9, 2020

(54) Device for estimating the moisture content and the availability of water in soils

(71) Applicant: Fernando SARRÍA PUEYO, SARRIGUREN (Navarra) (ES)

(72) Inventors: Fernando SARRÍA PUEYO, Navarra (ES); Amaia URIZ ZARZA, Navarra (ES); María de la Caridad PRADO SANTAMARÍA, Navarra (ES); Aurora ÁLVAREZ VIDAL, Navarra (ES); Miguel Ángel CAMPO BESCÓS, Navarra (ES); Rafael GIMÉNEZ DÍAZ, Navarra (ES)

(21) Appl. No.: 16/443,951

(22) Filed: Jun. 18, 2019

(30) Foreign Application Priority Data
Jun. 18, 2018 (ES) P201830592

(51) Int. Cl. G01N 33/24; A01G 25/16; G01N 27/22

(52) U.S. Cl. CPC G01N 33/246; G01N 27/221; G01N 27/223; A01G 25/167

(57) ABSTRACT

The present invention relates to an estimation device for estimating soil moisture content and water availability, comprising a casing (1) and soil moisture content and water availability estimation means (2, 3), and characterized in that said soil moisture content and water availability estimation means comprise at least a first sensor (2) detecting a first parameter of the water and at least a second sensor (3) detecting a second parameter of the water.

It allows the use of very economical materials and the result is a low-cost product. The fact that the production thereof may significantly lower product cost makes it affordable for farmers and allows the use of any number of devices suited to the cropped area.

The present invention relates to an estimation device for estimating soil moisture content and water availability, which allows detecting the need for soil irrigation.

### BACKGROUND OF THE INVENTION

The agricultural sector consumes 70% of worldwide water resources, and in Europe it consumes one third of all water reserves, although in southern European countries such as Spain this consumption increases up to 80% of all water resources. With the growing demand and climate change, current ecosystems that depend on water run the risk of suffering irreversible damage.

Water is a scarce resource, and excess water consumption represents a waste of resources (water catchment, storage, transport, and distribution) and increases energy consumption and water costs. Additionally, the world population is growing and resources are limited, so more efficient consumption is called for.

Farmers are faced with the need to cut down on water primarily for environmental reasons, but for economic reasons as well. The current trend is to monitor crop fields by means of measurement tools, such as weather stations and sensors detecting soil characteristics, among which moisture sensors stand out.

Nevertheless, these sensors are expensive, difficult to place and maintain, require periodic on-site monitoring by specialists, and since they are wired, the wiring cause problems, and they are also vulnerable to being stolen.

Excess irrigation leads primarily to environmental problems, such as:
Runoff: loss due to entrainment of nutrients in the crop field, which are always located in the superficial layers of soils. Soil erosion and transport causes degradation of both agricultural soils and the environment.
Seepage: this increases the movement of fertilizers below the root zone, transports particles of chemical elements (nitrates and phosphates) to drainage channels.
Salinization: water and other components transport salt to soils generating excess soluble salts which cause a loss of soil fertility.
Root asphyxiation: condition in a plant due to excess water in the roots.

The improper use of irrigation water, supplying larger amounts of water than that strictly needed for crop growth, fosters the nonpoint source pollution caused by farming on bodies of water, such as rivers, reservoirs, and aquifers. It is therefore possible that a sediment and nutrient supply in agricultural irrigation areas may end up reaching areas of impounded water, accelerating the growth of microorganisms and flora which translates into water quality degradation.

In some areas of Europe, the pollution caused by pesticides and fertilizers used in the farming constitutes in and of itself one of the main causes of deficient water quality.

On the other hand, insufficient irrigation reduces crop production.

In the last few decades, the market has offered several methods that intend to help farmers apply proper farming practices and make efficient use of water resources. However, their handling and installation complexity, as well as their high cost, have kept them far from mainstream.

However they are just now beginning to be developed, as technology enables it in relation to cost and development, and they are being strongly encouraged by the European Union. The systems that appear to have the most extensive application are the soil moisture sensors.

For example, patent document US2003015024A1 discloses a sensor detecting moisture in soils of any type by capacitance, by means of measuring changes in the dielectric constant of the material depending on the amount of water in the medium.

In turn, patent document US2009206853A1 discloses a cylindrical matrix of packed silica sand as the moisture transmission material, said matrix being contained by a non-fibrous, non-woven liner. Moisture from the soils goes through the silica sand and reaches two spaced apart electrodes which give a reading that is translated into the pressure whereby the soils retain the water, or in other words, it calculates the pressure whereby the root can absorb water.

Patent document US2017241973A1 discloses an objective, real-time measurable indicator for calculating distribution uniformity of water, salinity, conductivity, or temperature in soil. This portable compact analysis device has one or more sensors and a Global Positioning System (GPS) module configured for collecting location data and storing it in a server. However, in addition to being wired, it is very expensive and is designed for soils in golf courses.

Current moisture sensors are primarily limited, as a whole, to high-yield crop farms and research. Furthermore, the fact that they are connected by cable to a reader makes the handling thereof excessively complicated.

### DESCRIPTION OF THE INVENTION

The estimation device of the invention solves the aforementioned drawbacks and provides other advantages that will be described below.

The estimation device for estimating soil moisture content and water availability according to the present invention comprises a casing and soil moisture content and water availability estimation means, and is characterized in that said soil moisture content and water availability estimation means comprise at least a first sensor detecting a first parameter of the water and at least a second sensor detecting a second parameter of the water.

Advantageously, said at least a first sensor is arranged on the outside of said casing and is insulated from said casing by means of an impermeable barrier element.

Furthermore, said at least a second sensor is advantageously arranged on the inside of said casing.

Preferably, said casing is made of a porous material with the capacity to absorb liquids by capillarity, for example, red clay, such as fired red clay, or of a similar material in terms of strength, porosity, and capillarity.

Furthermore, said casing is preferably hollow, defining a spherical inner cavity, for example, though it could be of any other suitable shape.

According to a preferred embodiment, the first parameter detected by said at least a first sensor is the volume of water in a predetermined volume of the soils, and the second parameter detected by said at least a second sensor is soil moisture tension, i.e., the pressure-suction whereby water is retained by soils.

Furthermore, the estimation device for estimating soil moisture content and water availability according to the present invention also preferably comprises an electronic circuit located on the inside of said cavity of the casing, which advantageously comprises a wireless emitter.

The estimation device for estimating soil moisture content and water availability according to the present invention also advantageously comprises a battery located on the inside of said cavity of the casing.

Advantageously, said cavity is sealed, for example, by means of an epoxy resin or by means of a sealing gel or compound.

The device according to the present invention provides at least the following advantages:
Economical: The simplicity of its design allows the use of very economical materials, such as red clay, and electronics, for example, in Arduino, resulting in a low-cost product. The fact that the production thereof may significantly lower product cost makes it affordable for farmers and allows the use of any number of devices suited to the cropped area. p1 Hydrophilic property: Given its porosity, capillarity, and the behavior of its properties, red clay or similar materials is a material that imitates the ground or plant roots in terms of the capacity to absorb water by capillarity; it is therefore inferred that the coarse grain of the red clay provides the porosity needed to create a hydrophilic surface that meets the needs of the pressure sensor.
Material: The casing of the device is preferably made of red clay, a porous material that perfectly imitates the absorption properties of soils or plant roots since it is itself the same material, but firing it at 800° C. causes it to lose its plastic property, making the shape thereof irreversible upon contact with water, rendering it highly durable. It may be formed by other materials with the necessary properties of strength and water absorption by capillarity.
The same material, preferably clay, has a triple function. It provides the necessary strength to the probe, protects its internal elements, and emulates plant root behavior.
Unnecessary elements are eliminated: The casing acts like a support for the first sensor and like a container for the electronics housed on the inside of the casing, but it is an integral part itself of the second sensor.
Durability: The ceramic material fired at high temperatures provides high durability properties which, together with a rationed battery consumption, prolong its service life by at least 5 years, and it is therefore unnecessary to take it out every time a crop is planted. The needs of the crop itself will determine when to take it out.
Non-pollutant: All ground pollution is to be avoided, so it must be taken out at the end of the battery life and the possibility of replacing it studied. Nevertheless, the recovered material is about ⅔ biodegradable and the rest of the composition is recyclable.
Design: The spherical design of the device corresponds to the need for a simple and manageable installation that adapts homogenously to the ground to assure perfect contact with the soils under study, preventing the formation of air pockets that lead to mistaken readings. The result is a small spherical device which prevents burdensome methods.
Autonomy: An important objective is the elimination of cables, whereby a wireless radio signal is used, for example, thus achieving a high degree of autonomy. By means of the wireless radio signal, cables, readers, and batteries, which in addition to being uncomfortable are vulnerable to being stolen and the cause of accidents, are eliminated.
Technological simplicity: Programming of the device, for example, in Arduino, which is one of the simplest and most widely used languages, allows modifications in a simple manner, but what makes the system accessible is the simplicity in the real-time data reading that can be done by means of simple graphs in the farmer's device, thereby allowing the farmer to decide when to irrigate to maintain the level of moisture at suitable parameters. It can also be connected to automation and alert systems or other systems that may be considered helpful for this purpose.
Accessibility: Due to the simplicity in the installation, control, and management of the data and not needing to go anywhere to take readings as occurs with sensors on the market, the need for skilled labor is eliminated in irrigation crops in general and reduced in high-yield crop farms.
Precision and reliability: It resembles the sensors currently used in terms of precision in reproducing the read data.
Versatility: The installation of the device can be done by hand in a simple manner for intensive farming and in a less precise manner at the time of planting for extensive farming, thereby assuring that the devices are placed at the height of the roots where the read data is more precise.
Production optimization: Monitoring of soil water content is essential for helping farmers; with suitable irrigation a high production yield can be achieved because not only is the plant given enough water, root asphyxiation due to excess water is also avoided. The optimal yield, depending on the variety, has a range of specific water supply values. The probe thus designed provides this precision so as to achieve an optimal degree of production.
It allows finding out soil type. By means of the interpretation of the relationship between the two measurements (soil water amount and soil water pressure), referred to as the moisture retention curve, it is possible to find out if the ground is more clayey or sandy, with the former being the type that most retains water, and the latter being the type that most readily releases water.

### BRIEF DESCRIPTION OF THE DRAWINGS

To better understand the preceding explanation, a set of drawings is attached in which a practical embodiment is depicted schematically and only by way of non-limiting example.

FIG. 1 is an elevational view of the estimation device according to the present invention;

FIG. 2 is a cross-section view of the estimation device according to the present invention; and

FIG. 3 is a schematic view of the connection of the estimation device according to the present invention to a network, such as the Internet.

### DESCRIPTION OF A PREFERRED EMBODIMENT

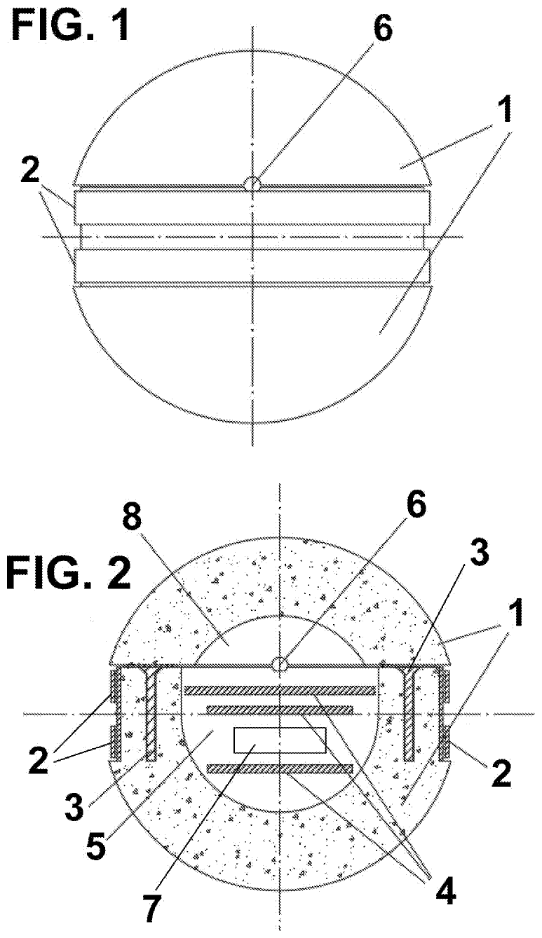

As shown in FIG. 1, the estimation device according to the present invention comprises a casing 1 comprising first sensors 2 and second sensors 3, for example, capacitance sensors, measuring two different parameters. For example, the first sensors 2 measure the crop soil water content or volume and the second sensors 3 measure the crop soil water pressure.

As can be seen, the first sensors 2 are placed on the outside of the casing 1, whereas the second sensors 3 are placed on the inside of the casing 1.

The material of the casing 1 has been selected based on two different functions; on one hand, it must be an integral part of the second sensors 3, and on the other, it is the container of each of the components.

According to one embodiment, the material of the casing 1 is red clay in this case which, due to its porosity, capillarity, and the behavior of its properties, is a suitable material for this purpose.

Furthermore, the red clay allows making a spherical casing 1 as a result of the plastic property of red clay 1, which allows molding the shape with detail. By means of firing at 800° C. for 8 hours, the clay loses its plastic property, making the shape thereof irreversible upon contact with water, rendering it highly durable.

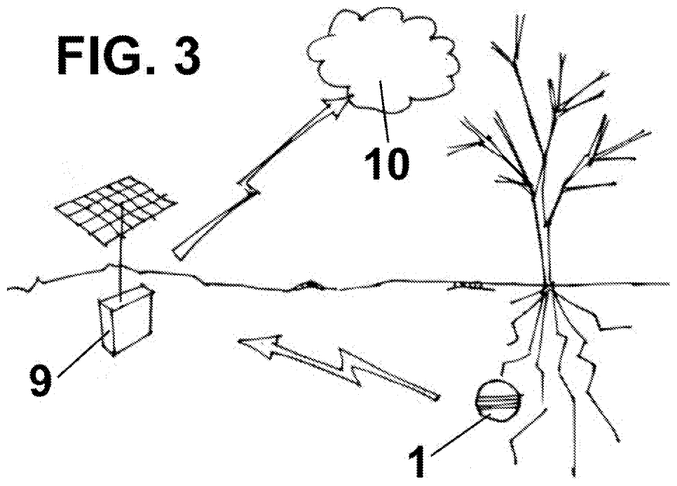

Red clay behaves like a moisture transmission material and its coarse grain has a size suitable for imitating the ground in terms of the capacity to absorb water, thereby providing a hydrophilic material. By not reaching 900° C. during firing, crystallization of the silica that are found in the clay composition and render the material impermeable, is avoided. The second sensors 3, placed on the inside of the casing 1, as seen in FIG. 2, can therefore detect the soil water pressure due to the properties of the material of the casing 1.

The thickness of the walls of the casing 1 is about 2 between and 2.5 cm according to calculated data depending on the porous material of the casing 1, and the second sensors 3 are to be housed therein, said sensors comprising two poles, preferably in parallel positions and spaced apart 0.9 cm, providing a reading of the presence of moisture that has seeped by capillarity into the casing 1.

The first sensors 2 are located on the outside of the casing 1 and in direct contact with the cropped soils. The first sensors 2 preferably comprise two conductive poles in parallel positions and spaced apart, for example, 0.9 cm, providing a reading of the presence of moisture on the outside of the casing 1.

It should be indicated that the first sensors are not to take readings of the water contained in the casing 1. Accordingly, to prevent contact of the first sensors 2 with the casing 1, the latter comprises an insulating impermeable barrier 6 against the capacitance signal, for example, a water-resistant resin. This barrier 6 also fulfills the purpose of water circulating into the casing 1, respecting the measurements calculated for correct operation of the second sensors 3.

The first and second sensors 2, 3 are capable of measuring the dielectric constant of the soils, and thereby determining the levels of volumetric moisture present at all times. By means of electronic circuits 4 located in an internal cavity 5 of the casing 1 and powered by a battery 7, the analog signal supplied by the first and second sensors 2, 3 will be translated into the values that correspond with the magnitude to be measured; for the second sensors 3 which measure the pressure, the signal will be translated into centibars (cbar) of pressure, and for the first sensors 2 which measure the volume, the signal will be translated into percentage (%) by volume.

To completely insulate the electronic circuits 4 inside the casing 1 and protect them from moisture, corrosion, blows, and vibrations, the cavity 5 is filled with an encapsulating gel compound that assures sealing and tightness.

As can be seen in FIG. 2, the casing 1 is formed by a larger and a smaller segment attached to one another. After assembling each of the parts of the estimation device in the larger segment of the casing 1, it is hermetically closed, placing the smaller segment and sealing both segments with an epoxy resin seal 8, for example.

The operation of the estimation device according to the present invention is very simple, as it merely requires being installed in crop soils in which the amount of water is to be detected.

The first and second sensors 2, 3 measure the parameters described above, i.e., the water pressure and volume, and this information is sent in a wireless manner by means of electronic circuits 4 to external control means 9, for example, powered by solar energy.

Said external control means 9 can be, for example, a computer, or a gateway connected to the Internet 10, such that the person in charge of crop soils can consult the information provided by a plurality of detection devices according to the present invention and make the necessary decisions with respect to the need to irrigate crop soils or not.

Although reference has been made to a specific embodiment of the invention, it shall be evident to one skilled in the art that the described estimation device is susceptible to a number of variations and modifications, and that all the mentioned details can be replaced with other technically equivalent details without departing from the scope of protection defined by the appended claims.

1. Estimation device for estimating soil moisture content and water availability, comprising a casing and soil moisture content and water availability estimation means, wherein that said soil moisture content and water availability estimation means comprise at least a first sensor detecting a first parameter of the water and at least a second sensor detecting a second parameter of the water.

2. Estimation device for estimating soil moisture content and water availability according to claim 1, wherein said at least a first sensor is arranged on the outside of said casing.

3. Estimation device for estimating soil moisture content and water availability according to claim 1, wherein said at least a first sensor is insulated from said casing by means of an impermeable barrier element.

4. Estimation device for estimating soil moisture content and water availability according to claim 1, wherein said at least a second sensor is arranged on the inside of said casing.

5. Estimation device for estimating soil moisture content and water availability according to claim 1, wherein said casing is made of a porous material.

6. Estimation device for estimating soil moisture content and water availability according to claim 5, wherein said casing is made of red clay.

7. Estimation device for estimating soil moisture content and water availability according to claim 6, wherein said casing is made of fired red clay.

8. Estimation device for estimating soil moisture content and water availability according to claim 1, wherein said casing is hollow, defining an inner cavity.

9. Estimation device for estimating soil moisture content and water availability according to claim 1, wherein said casing is spherical.

10. Estimation device for estimating soil moisture content and water availability according to claim 1, wherein the first parameter detected by said at least a first sensor is the volume of water in a predetermined volume of the soil.

11. Estimation device for estimating soil moisture content and water availability according to claim 1, wherein the second parameter detected by said at least a second sensor is the pressure exerted by soil water.

12. Estimation device for estimating soil moisture content and water availability according to claim 8, which further comprises an electronic circuit located on the inside of said cavity of the casing.

13. Estimation device for estimating soil moisture content and water availability according to claim 12, wherein said electronic circuit comprises a wireless emitter.

14. Estimation device for estimating soil moisture content and water availability according to claim 8, which also comprises a battery located on the inside of said cavity of the casing.

15. Estimation device for estimating soil moisture content and water availability according to claim 8, wherein said cavity is sealed.
